SEQUENCE LISTING (9 sequences)

SEQ ID NO: 1 - protein, 16 aa, Unknown
  PHOSPHORYLATION (3)...(3): Sequences harboring phosphorylation sites.
FQSEEQQQTEDELQDK

SEQ ID NO: 2 - protein, 13 aa, Unknown
  PHOSPHORYLATION (11)...(11): Sequences harboring phosphorylation sites.
ASGQAFELILSPR

SEQ ID NO: 3 - protein, 14 aa, Unknown
  PHOSPHORYLATION (11)...(11): Sequences harboring phosphorylation sites.
SKESVPEFPLSPPK

SEQ ID NO: 4 - protein, 14 aa, Unknown
  PHOSPHORYLATION (4)...(4): Sequences harboring phosphorylation sites.
LPSSPVYEDAASFK

SEQ ID NO: 5 - protein, 16 aa, Unknown
  PHOSPHORYLATION (3)...(3)
  PHOSPHORYLATION (7)...(7): Sequences harboring phosphorylation sites.
TQTPPVSPAPQPTEER

SEQ ID NO: 6 - protein, 8 aa, Unknown
  PHOSPHORYLATION (3)...(3): Sequences harboring phosphorylation sites.
IESPKLER

SEQ ID NO: 7 - protein, 17 aa, Unknown
  UNSURE (2)...(2)
  PHOSPHORYLATION (12)...(12): Sequences harboring phosphorylation sites.
VMHTQCHSTPDSAEDVR

SEQ ID NO: 8 - protein, 16 aa, Unknown
  PHOSPHORYLATION (10)...(10): Sequences harboring phosphorylation sites.
MRDWVIPPISCPENEK

SEQ ID NO: 9 - protein, 16 aa, Unknown
  PHOSPHORYLATION (5)...(5): Sequences harboring phosphorylation sites.
ELILSPRSKESVPEFP

(19) United States
(12) Patent Application Publication
(10) Pub. No.: US 2007/0099250 A1
(43) Pub. Date: May 3, 2007

(54) Methods for treating proliferative diseases and for monitoring the effectiveness of treatment of proliferative diseases

(76) Inventors: Ping Hu, Summit, NJ (US); Yingqi Karen Wang, East Hanover, NJ (US); David Bryant Batt, Wayland, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/553,091

(22) PCT Filed: Apr. 13, 2004
(86) PCT No.: PCT/EP04/03877

§ 371 Date: Nov. 27, 2006

Related U.S. Application Data
(60) Provisional application No. 60/462,723, filed on Apr. 14, 2003.

(51) Int. Cl. G01N 33/574

(52) U.S. Cl. 435/7.23

(57) ABSTRACT

The present invention relates to phosphoproteins useful as biomarkers for identifying and treating patients suffering from diseases characterized by an aberrant MAP kinase signaling pathway, for example proliferative diseases like certain cancers, monitoring the efficacy of treatment of patients having the disease by administering Raf kinase inhibitors and diagnosing the disease in patients.

### BACKGROUND OF THE INVENTION

1. Field of the Invention

The present invention relates to phosphoproteins useful as biomarkers for identifying and treating patients suffering from diseases characterized by an aberrant MAP kinase signaling pathway, for example proliferative diseases like certain cancers, monitoring the efficacy of treatment of patients having the disease by administering Raf kinase inhibitors and diagnosing the disease in patients.

2. Description of the Related Art

Cells communicate various aspects of their extracellular environment to the nucleus by using various signal transduction pathway. Many of these signals are transmitted by protein kinases which activate various factors through the transfer of phosphate groups. Disruption of signal transduction by inhibiting appropriate kinase activity can have a clinical benefit as demonstrated by imatinib, an inhibitor of ber-abl kinase, which is marketed under the brand name Gleevec (in the United States).

The MAP kinase signaling pathway is known in the art as one of the pathways for growth factors to transmit their signal to proliferate from the extracellular environment to the cell nucleus. The growth factors activate transmembrane receptors located on the cell surface which in turn start a cascade whereby RAS, a G-protein, is activated and binds to Raf kinase; a serine/threonine kinase, with high affinity and causes its translocation to the cell membrane where Raf activation takes place. Activated Raf then phosphorylates and activates Mitogen-Activated Protein Kinase Kinase (MEK), which in turn phosphorylates and activates the extracellular-signal-regulated protein kinase (ERK). Activated ERK phosphorylates cytoplasmic targets and translocates to the nucleus where it activates various transcription factors.

The RAF kinase family is known to have three members designated C-RAF, also known as RAF-1, B-RAF and A-RAF. It has been reported that B-RAF kinase is commonly activated by one of several somatic point mutations in human cancer, including 59% of the melanoma cell lines tested. See Davies et al., Nature, Vol.417, pp. 949-954 (2002). Efficient inhibitors of RAF kinase, particular C-RAF kinase and wild and mutated B-RAF kinase, particular the V599E mutant B-RAP kinase are disclosed herein and have been previously described in U.S. Published Application 2002-0010191.

The RAF kinase inhibitors are useful as therapeutic agents for the treatment for proliferative diseases characterized by an aberrant MAP kinase signaling pathway, particularly many cancers characterized by deregulated/hyperactive MAPK pathway, or an activating mutation of RAF kinase, such as melanoma having mutated B-RAF, especially wherein the mutated B-RAF is the V599E mutant.

While knowledge of the aforementioned upstream kinases of the MAPK pathway has greatly increased, less is known about downstream target phosphoproteins regulated by the MAP kinase pathway. The identification of such downstream phosphoproteins and the phosphorylation state of these phosphoproteins, i.e., the level of phosphorylation of the proteins, in response to therapeutic agents such as Raf kinase inhibitors are important in demonstrating that the MAP kinase pathway is appropriately modulated by the therapeutic agent. Downstream target phosphoproteins and their phosphorylation state in response to therapeutic agents can also be used as biomarkers for identifying and treating a disease such as cancer, diagnosing and monitoring progression of and improvements in a disease, and for clinically evaluating whether a therapeutic agent successful blocks or activates a target.

Accordingly, there is a need in the art to identify downstream phosphoproteins regulated by the MAP kinase pathway and the phosphorylation state of these phosphoproteins in response to therapeutic agents, such as Raf kinase inhibitors.

### BRIEF DESCRIPTION OF THE DRAWINGS

FIG. 1. Inverse Labeling-MS analysis of HCT1 16 cell lysate treated with Raf kinase inhibitor 1-(4-t-butylanilino)-4-[(pyridin-4-yl)-methyl]-isoguinoline (BPMI). Down-regulation of Op18 Ser²⁵ phosphorylation is detected upon BPMI treatment.

FIG. 2. (A) Direct LC/MS analysis of lysate from tumor tissue DU145 treated with Raf kinase inhibitor BPMI (* mouse serun albumin; ° mouse hemoglobin); and (B) Methyl esterification/IMAC removed blood contaminants; down-regulation of OP18 Ser²⁵ phosphorylation is confirmed in vivo.

### DESCRIPTION OF THE INVENTION

All patent applications, patents and literature references cited herein are hereby incorporated by reference in their entirety.

The term “effective amount” refers to an amount of a Raf kinase inhibitor such as BPMI, which, when administered to the patient, is effective to treat a disease characterized by an aberrant MAP kinase signaling pathway for example a proliferative disease such as cancer. With respect to treatment of a proliferative disease this includes a reduction of symptoms of the disease, a shrinking of tumor size, death of the cells of the proliferative disease (cancer), and any other indicators known in the art which show the treatment of the proliferative disease.

The present invention relates to the identification of phosphoproteins, oncoprotein 18 (Op18), oncogene EMS1 and heat-shock 110 kD protein (see Table 1) in tumor cells, in which phosphorylation is down-regulated upon treatment with a Raf kinase inhibitor. In particular, phosphorylation of the serine 25 residue of Op18 is down-regulated upon treatment of tumor cells comprising Op18 with a Raf kinase inhibitor and the change in Op18 serine 25 phosphorylation correlates quantitatively with the change in phosphorylation state of MEK. These phosphoproteins and the phosphorylation state of these phosphoproteins in response to a Raf kinase inhibitor can be utilized as biomarkers for: 1) identifying subjects having or at risk of developing a disease characterized by an aberrant MAP kinase signaling pathway, e.g., a proliferative disease such as cancer, and then treating the subjects having or at risk of developing the disease with Raf kinase inhibitors; 2) evaluating the efficacy of a Raf kinase inhibitor in treating the disease; and 3) diagnosing the disease in a patient and monitoring the progress of such patients.

According in one aspect, a method is provided for treating a patient having a disease or at risk of developing a disease characterized by an aberrant MAP kinase signaling pathway. The method comprises:
a) identifying a patient suffering from the disease or at risk of developing the disease by measuring an increased level of phosphorylation of at least one phosphoprotein identified in Table 1 in a biological sample obtained from the patient; and
b) administering to the patient an effective amount of a Raf kinase inhibitor.

In general, the disease characterized by an aberrant MAP kinase signaling pathway is a proliferative disease, particularly a cancer characterized by increased RAF kinase activity, for example one which overexpresses wild-type B- or C-RAF kinase, or that expresses an activating mutant RAF kinase, e.g., a mutant B-RAF kinase. Cancers wherein a mutated RAF kinase has been detected include melanoma, colorectal cancer, ovarian cancer, gliomas, adenocarcinomas, sarcomas, breast cancer, lung cancer and liver cancer. Mutated B-RAF kinase is especially prevalent in many melanomas.

In accordance with the present invention, a biological sample is taken from the patient, for example, as a result of a biopsy or resection, and tested to determine whether at least one of the phosphoproteins identified in Table 1 or 2 exhibit an increased level of phosphorylation which is indicative that the patient has or is at risk of developing the disease. The biological sample can be obtained from a cell or cells, a tissue or tissues, blood, serun, stool, urine, sputum, amniotic fluid or a bone tissue biopsy. An increased level of phosphorylation of the phosphoprotein in the biological sample, e.g., a tumor tissue, can be detected by comparing the level of phosphorylation of the biological sample with the level of phosphorylation of the phosphoprotein in a normal sample of the tissue obtained from the same individual or from a disease-free subject. The sample obtained from the disease-free subject can be obtained at the same time as the test sample obtained from the subject, or can be a preestablished control for which the level of phosphorylation of the protein was determined at an earlier time.

The level of phosphorylation of the phosphoprotein(s) in the test and normal samples can be determined by technique will known in the art, e.g., by labeling the samples with ³²Pi and performing immunoprecipitation or western blot analysis of the protein(s) utilizing antibodies specific for the protein(s). The level of phosphorylation of the protein in the two samples can also be determined by mass spectrometry using an inverse labeling method as described in U.S. Published Application Ser. No.2002-0090652 (see also Examples 2 and 3).

In a preferred embodiment, the level of phosphorylation of serine 25 residue or serine 3 8 residue of the phosphoprotein, Op18, is measured and detected by well known methods.

Upon detecting an increased level of phosphorylation of the phosphoprotein in the test sample of the patient suspected of having the disease or of developing the disease compared with the normal sample from the disease-free subject, a Raf kinase inhibitor is then administered to the patient. Administration of the Raf kinase inhibitor results in a down-regulation of the level of phosphorylation of the phosphoprotein. Down-regulation of the phosphorylation state of the phosphoprotein can be detected in a subsequent test sample obtained from the patient to monitor the efficacy of the treatment as described below.

In a particularly useful embodiment, the Raf kinase inhibitor is a compound of the formula (I) 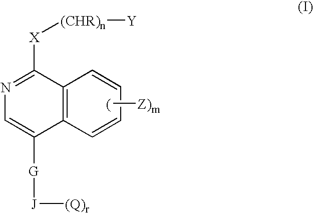
wherein

r is from 0-2;

n is from 0-2;

m is from 0-4;

G is a direct bond, lower alkylene, —CH₂—O—, —CH₂—S—, —CH₂—NH—, —SO2—, oxa (—O—), thia (—S—) or —NR—, or is lower alkylene substituted by acyloxy, oxo, halogen or hydroxy.

J is aryl, heteroaryl, cycloalkyl or heterocycloalkyl, wherein aryl is an aromatic radical having from 6-14 carbon atoms, such as phenyl, naphthyl, fluorenyl and phenanthrenyl;
heteroaryl is an aromatic radical having from 4-14, especially from 5-7 ring atoms, of which 1, 2 or 3 atoms are chosen independently from N, S and O, such as furyl, pyranyl, pyridyl, 1,2-, 1,3- and 1 ,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofiranyl, chromenyl, purinyl, thianthrenyl, xanthenyl, acridinyl, carbazolyl and phenazinyl;
cycloalkyl is a saturated cyclic radical having from 3-8, preferably from 5-6 ring atoms, such as cyclopropyl, cyclopentyl and cyclohexyl;
heterocycloalkyl is a saturated cyclic radical having from 3-8, preferably from 5-6 ring atoms, of which 1, 2 or 3 atoms are chosen independently from N, S and O, such as piperidyl, piperazinyl, imidazolidinyl, pyrrolidinyl and pyrazolidinyl;

Q is a substituent on 1 or 2 carbon atoms selected from the group consisting of halogen, unsubstituted or substituted lower alkyl, —OR₂, —SR₂, —NR2, —NRS(O)2N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁₋₄-alkyl-aryl, —C₁₋₄alkyl-heteroaryl, —C₁₋₄alkyl-heterocyclyl, amino, mono- or di-substituted amino;

R is H or lower alkyl;

R₂ is unsubstituted or substituted alkyl, unsubstituted or substituted cycloalkyl, phenyl, —C₁₋₄alkyl-aryl, —C₁₋₄alkyl-heteroaryl or —C₁₋₄alkyl-heterocycloalkyl;

X is Y, —N(R)—, oxa, thio, sulfone, sulfoxide, sulfonamide, amide, or ureylene, preferably —NH—;

Y is H, lower alkyl, substituted or unsubstituted aryl, substituted or unsubstituted heteroaryl, substituted or unsubstituted cycloalkyl or substituted or unsubstituted heterocycloalkyl; and

Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m>2), the substituents Z are identical or different;

or an N-oxide of the mentioned compound, wherein one or more N atoms carry an oxygen atom;

or a pharmaceutically acceptable salt thereof.

The compounds of formula (I) inhibit RAF kinase and have pharmaceutical utility based on this property.

Within the context of the present disclosure, the general terms used hereinbefore and hereinafter preferably have the following meanings, unless indicated otherwise.

The term “lower” denotes a radical having up to and including a maximum of 7, especially up to and including a maximum of 4 carbon atoms, the radicals in question being unbranched or branched one or more times.

Any reference to compounds, salts and the like in the plural is always to be understood as including one compound, one salt or the like.

Asymmetric carbon atoms which may be present, e.g., in compounds of formula (I) (or an N-oxide thereof), wherein n=1 and R is lower alkyl; may have the (R), (S) or (R,S) configuration, preferably the (R) or (S) configuration. Substituents at a double bond or a ring may be in the cis (=Z) or trans (=E) form. Accordingly, the present compounds may be in the form of isomeric mixtures or in the form of pure isomers, preferably in the form of an enantiomerically pure diastereoisomer.
The index r is preferably 0 or 1. It may also be 2.
The index n is preferably 0 or 1, especially 0. It may also be 2.
The index m is preferably 0, 1 or 2, especially 0, or also 1.
Preferably, J is heteroaryl containing at least one, but not more than three N.

Lower alkyl is especially C₁₋₄alkyl, e.g., n-butyl, sec-butyl, tert-butyl, n-propyl, isopropyl or, especially, methyl or also ethyl, or, in the case of Y as lower alkyl, it may be especially isopentyl.

Aryl is preferably an aromatic radical having from 6-14 carbon atoms, especially phenyl, naphthyl, fluorenyl or phenanthrenyl, the mentioned radicals being unsubstituted or substituted by one or more substituents, preferably up to three, especially one or two substituents, especially selected from amino; mono- or di-substituted amino; halogen; alkyl; substituted alkyl; hydroxyl; etherified or esterified hydroxyl; nitro; cyano; carboxy; esterified carboxy; alkanoyl; carbamoyl; N-mono- or N,N-di-substituted carbamoyl; amidino; guanidine; mercapto; sulfo; phenylthio; phenyl-lower alkylthio; alkylphenylthio; phenylsulfinyl; phenyl-lower alkylsulfinyl; alkylphenylsulfinyl; phenylsulfonyl; phenyl-lower alkanesulfonyl; alkylphenylsulfonyl; lower alkenyl, such as ethenyl and phenyl; lower alkylthio, such as methylthio; lower alkanoyl, such as acetyl; lower alkylmercapto, such as methylmercapto (—S—CH₃); halo-lower alkylmercapto, such as trifluoromethylmercapto (—S—CF₃); lower alkanesulfonyl; halo-lower alkanesulfonyl, such as, especially, trifluoromethanesulfonyl, dihydroxybora (—B(OH)₂) and heterocyclyl; and lower alkylenedioxy, such as methylenedioxy, bonded to adjacent carbon atoms of the ring; aryl is preferably phenyl that is unsubstituted or substituted by one or two identical or different substituents from the group consisting of amino; lower alkanoylamino, especially acetylamino; halogen, especially fluorine, chlorine or bromine; lower alkyl, especially methyl, or also ethyl or propyl; halo-lower alkyl, especially trifluoromethyl; hydroxy; lower alkoxy, especially methoxy, or also ethoxy; phenyl-lower alkoxy, especially benzyloxy; and cyano, or (alternatively or additionally to the preceding group of substituents) C₈₋₁₂alkoxy, especially n-decyloxy; carbamoyl; lower alkylcarbamoyl, such as N-methyl- or N-tert-butyl-carbamoyl; lower alkanoyl, such as acetyl or phenyloxy; halo-lower alkyloxy, such as trifluoromethoxy or 1,1,2,2-tetrafluoroethyloxy; lower alkoxycarbonyl, such as ethoxycarbonyl; lower alkylmercapto such as methylmercapto; halo-lower alkylmercapto, such as trifluoromethylmercapto; hydroxy-lower alkyl, such as hydroxymethyl or 1-hydroxymethyl; lower alkanesulfonyl, such as methanesulfonyl; halo-lower alkanesulfonyl, such as trifluoromethanesulfonyl, phenylsulfonyl, dihydroxybora (—B(OH)2), 2-methyl-pyrimidin-4-yl, oxazol-5-yl, 2-methyl-1,3-dioxolan-2-yl, 1H-pyrazol-3-yl or 1-methyl-pyrazol-3-yl; and lower alkylenedioxy, such as methylenedioxy, bonded to two adjacent carbon atoms, more especially by one or two identical or different substituents selected from lower alkyl, especially methyl; halogen, especially chlorine or bromine; and halo-lower alklyl, especially trifluoromethyl. Aryl is preferably also naphthyl.

Heteroaryl is preferably an unsaturated heterocyclic radical in the bonding ring and is preferably mono- or also bi- or tri-cyclic; wherein at least in the ring bonding to the radical of the molecule of formula (I) one or more, preferably from 1-4, especially 1 or 2 carbon atoms of a corresponding aryl radical have been replaced by a hetero atom selected from the group consisting of nitrogen, oxygen and sulfur, the bonding ring having preferably from 4-14, especially from 5-7 ring atoms; wherein heteroaryl is unsubstituted or substituted by one or more, especially from 1-3, identical or different substituents from the group consisting of the substituents mentioned above as substituents of aryl; and is especially a heteroaryl radical selected from the group consisting of imidazolyl, thienyl, firyl, pyranyl, thianthrenyl, isobenzofuranyl, benzofuranyl, chromenyl, 2H-pyrrolyl, pyrrolyl, lower alkyl-substituted imidazolyl, benzimidazolyl, pyrazolyl, thiazolyl, isothiazolyl, oxazolyl, isoxazolyl, pyridyl, pyrazinyl, pyrimidinyl, pyridazinyl, indolizinyl, isoindolyl, 3H-indolyl, indolyl, indazolyl, triazolyl, tetrazolyl, purinyl, 4H-quinolizinyl, isoquinolyl, quinolyl, phthalazinyl, naphthyridinyl, quinoxalyl, quinazolinyl, cinnolinyl, pteridinyl, carbazolyl, phenanthridinyl, acridinyl, perimidinyl, phenanthrolinyl and furazanyl, each of those radicals being bonded via a ring having at least one hetero atom to the radical of the molecule of formula (I); pyridyl is especially preferred. Special preference is given also to indolyl that is substituted by halogen, especially by fluorine, especially 6-fluoroindol-3-yl.

Heteroaryl is especially a 5- or 6-membered aromatic heterocycle having 1 or 2 hetero atoms selected from the group consisisting of nitrogen, oxygen and sulfur, which heterocycle may be unsubstituted or substituted, especially by lower alkyl, such as methyl; preference is additionally given to a radical selected from 2-methyl-pyrimidin4-yl, 1H-pyrazol-3-yl and 1-methyl-pyrazol-3-yl.

Heterocycloalkyl is especially a saturated 5- or 6-membered heterocycle having 1 or 2 hetero atoms selected from the group consisting of nitrogen, oxygen and sulfur, which heterocycle may be unsubstituted or substituted, especially by lower alkyl, such as methyl; preference is given to a radical selected from oxazol-5-yl and 2-methyl-1,3-dioxolan-2-yl.

Mono- or di-substituted amino is especially amino that is substituted by one or two identical or different radicals from lower alkyl, such as methyl; hydroxy-lower alkyl, such as 2-hydroxyethyl; phenyl-lower alkyl; lower alkanoyl, such as acetyl; benzoyl; substituted benzoyl, wherein the phenyl radical is unsubstituted or, especially, is substituted by one or more, preferably one or two, substituents selected from nitro and amino, or also from halogen, amino, N-lower alkylamino, N,N-di-lower alkylamino, hydroxy, cyano, carboxy, lower alkoxycarbonyl, lower alkanoyl and carbamoyl; and phenyl-lower alkoxycarbonyl wherein the phenyl radical is unsubstituted or, especially, is substituted by one or more, preferably one or two, substituents selected from nitro and amino, or also from halogen, amino, N-lower alkylamino, N,N-di-lower alkylamino, hydroxy, cyano, carboxy, lower alkoxycarbonyl, lower alkanoyl and carbamoyl; and is preferably N-lower alkylamino, such as N-methylamino or hydroxy-lower alkylamino, such as 2-hydroxyethylamino; phenyl-lower allylamino, such as benzylamino, N,N-di-lower alkylamino, N-phenyl4ower alkyl-N-lower alkylamino or N,N-di-lower alkylphenylamino; lower alkanoylamino, such as acetylamino; or a substituent selected from the group consisting of benzoylamino and phenyl-lower alkoxycarbonylamino, wherein in each case the phenyl radical is unsubstituted or, especially, is substituted by nitro or amino, or also by halogen, amino, N-lower alkylamino, N,N-di-lower alkylamino, hydroxy, cyano, carboxy, lower alkoxycarbonyl, lower alkanoyl or by carbamoyl, or alternatively or additionally to the preceding group of radicals, by aminocarbonylamino.

Halogen is especially fluorine, chlorine, bromine or iodine, more especially fluorine, chlorine or bromine, in particular fluorine and chlorine.

Alkyl has preferably up to a maximum of 12 carbon atoms and is especially lower alkyl, more especially methyl, or also ethyl, n-propyl, isopropyl or tert-butyl.

Substituted alkyl is alkyl as last defined, especially lower alkyl, preferably methyl, which may contain one or more, especially up to 3 substituents, selected especially from the group consisting of halogen, especially fluorine, and also amino, N-lower alkylamino, N,N-di-lower alkylamino, N-lower alkanoylamino, hydroxy, alkoxy, cyano, carboxy, lower alkoxycarbonyl and phenyl-lower alkoxycarbonyl. Trifluoromethyl is an important substituted alkyl.

Etherified hydroxy is especially C₈₋₂₀alkyloxy, such as n-decyloxy; lower alkoxy (preferred), such as methoxy, ethoxy, isopropyloxy or n-pentyloxy; phenyl-lower alkoxy, such as benzyloxy or also phenyloxy; or, alternatively or additionally to the preceding group, C₈₋₂₀alkyloxy, such as n-decyloxy; halo-lower alkoxy, such as trifluoromethyloxy or 1,1,2,2-tetrafluoroethoxy.

Esterified hydroxy is especially lower alkanoyloxy, benzoyloxy, lower alkoxycarbonyloxy, such as tert-butoxycarbonyloxy; or phenyl-lower alkoxycarbonyloxy, such as benzyloxycarbonyloxy.

Esterified carboxy is especially lower alkoxycarbonyl, such as tert-butoxycarbonyl or ethoxycarbonyl, phenyl-lower alkoxycarbonyl or phenyloxycarbonyl.

Alkanoyl is especially alkyl-carbonyl, more especially lower alkanoyl, e.g., acetyl.

N-Mono- or N,N-di-substituted carbamoyl is especially substituted at the terminal nitrogen by one or two substituents lower aIkyl, phenyl4ower alkyl or hydroxy-lower alkyl.

Alkylphenylthio is especially lower alkylphenylthio.

Alkyphenylsulfinyl is especially lower alkylphenylsulfinyl.

Alkyiphenylsulfonyl is especially lower alkylphenylsulfonyl.

Pyridyl Y is preferably 3- or 4-pyridyl.

Unsubstituted or substituted cycloalkyl is preferable C₃₋₈cycloalkyl, which is unsubstituted or is substituted in the same manner as aryl, especially as defined for phenyl. Preference is given to cyclohexyl, or also cyclopentyl or cyclopropyl. Preference is given also to 4-lower alkyl-cyclohexyl, such as 4-tert-butylcyclohexyl.

If present, Z is preferably amino; hydroxy-lower alkylamino, such as 2-hydroxyethylamino; lower alkanoylamino, such as acetylamino; nitrobenzoylamino, such as 3-nitrobenzoylarnino; aminobenzoylamino, such as 4-aminobenzoylamino; phenyl-lower alkoxycarbonylamino, such as benzyloxycarbonylamino; or halogen, such as bromine; preferably only one substituent is present (m=1), especially one of the last-mentioned substituents, especially halogen. Very special preference is given to a compound of formula (I), or an N-oxide thereof, wherein Z is not present (m=0).

G is preferably a direct bond (i.e. a bond directly between J and the ring) or methylene.

Aryl in the form of phenyl that is substituted by lower alkylenedioxy, such as methylenedioxy, bonded to two adjacent carbon atoms is preferably 3,4-methylenedioxyphenyl.

An N-oxide of a compound of formula (I) is preferably an N-oxide in which an isoquinoline ring nitrogen or a nitrogen in the J moiety carries an oxygen atom, or more than one of the mentioned nitrogen atoms carry an oxygen atom.

Salts are especially the pharmaceutically acceptable salts of compounds of formula (I), or an N-oxide thereof.

Such salts are formed, e.g., by compounds of formula (I), or an N-oxide thereof, having a basic nitrogen atom as acid addition salts, preferably with organic or inorganic acids, especially the pharmaceutically acceptable salts. Suitable inorganic acids are, e.g., hydrohalic acids, such as hydrochloric acid (HCl); sulfuric acid; or phosphoric acid. Suitable organic acids are, e.g., carboxylic phosphonic, sulfonic or sulfamic acids, e.g., acetic acid; propionic acid; octanoic acid; decanoic acid; dodecanoic acid; glycolic acid; lactic acid; 2-hydroxybutyric acid; gluconic acid; glucosemonocarboxylic acid; fumaric acid; succinic acid; adipic acid; pimelic acid; suberic acid; azelaic acid; malic acid; tartaric acid; citric acid; glucaric acid; galactaric acid; amino acids, such as glutamic acid, aspartic acid, N-methylglycine, acetylaminoacetic acid, N-acetylasparagine, N-acetylcysteine, pyruvic acid, acetoacetic acid, phosphoserine, 2- or 3-glycerophosphoric acid, maleic acid, hydroxymaleic acid, methylmaleic acid, cyclohexanecarboxylic acid, benzoic acid, salicylic acid, 1- or 3-hydroxynaphthyl-2-carboxylic acid, 3,4,5-trimethoxybenzoic acid, 2-phenoxybenzoic acid, 2-acetoxybenzoic acid, 4aminosalicylic acid, phthalic acid, phenylacetic acid, glucuronic acid, galacturonic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalenedisulfonic acid, N-cyclohexylsulfamic acid or N-methyl-, N-ethyl- or N-propyl-sulfamic acid; or other organic protonic acids, such as ascorbic acid.

When negatively charged radicals, such as carboxy or sulfo, are present, salts with bases can also be formed, e.g., metal or ammonium salts, such as alkali metal; alkaline earth metal salts, e.g., sodium, potassium, magnesium or calcium salts; ammonium salts with ammonia or suitable organic amines, such as tertiary monoamines, e.g., triethylamine or tri(2-hydroxyethyl)amine; or heterocyclic bases, e.g., N-ethylpiperidine or N,N′-dimethyl-piperazine.

When a basic group and an acid group are present in the same molecule, a compound of formula (I), or an N-oxide thereof, can also form internal salts.

For isolation or purification it is also possible to use pharmaceutically unacceptable salts, e.g., picrates or perchlorates. Only the pharmaceutically acceptable salts or the free compounds, optionally in the form of pharmaceutical compositions, are used therapeutically, and those are therefore preferred.

In view of the close relationship between the novel compounds in free form and in the form of their salts, including also those salts which can be used as intermediates, e.g., in the purification of the novel compounds or for their identification, hereinbefore and hereinafter any reference to the free compounds is also to be understood as including the corresponding salts, as appropriate and expedient.

Lower alkylene G may be branched or, preferably, unbranched and is especially branched or, preferably, unbranched C₁-C₄alkylene, especially methylene (—CH2—), ethylene (—CH₂—CH₂—), trimethylene (—CH₂—CH₂—CH₂—) or tetramethylene (—CH₂—CH₂—CH₂—CH₂—). G is preferably methylene. Lower alkylene G is preferably unsubstituted, but may be substituted by acyloxy, oxo, halogen or hydroxy.

A compound of formula (I) (or an N-oxide thereof) can be administered on its own or in combination with one or more other therapeutic agents, it being possible for fixed combinations to be used or for the disclosed compound and one or more other therapeutic agents to be administered in a staggered manner over time or independently of one another, or the combined administration of fixed combinations and of one or more other therapeutic agents is possible. In particular, the administration of a compound of formula (I) (or an N-oxide thereof) for tumor treatment can be carried out, alongside or additionally, in combination with chemotherapy (combination with one or more other chemotherapeutic agents, especially cytostatics, or with hormones or compounds having a hormone-like activity), radiotherapy, immunotherapy, surgical treatment or combinations thereof. Long-term therapy is also possible, as is adjuvant therapy in conjunction with other treatment methods, such as those just mentioned. Treatment to maintain the status of a patient after tumor remission or even chemopreventive treatment, e.g., in the case of at-risk patients, is also possible.

There come into consideration as therapeutic agents with which the disclosed compounds can be combined especially one or more antiproliferative, cytostatic or cytotoxic compounds, e.g., one or more chemotherapeutic agents selected from the group comprising an inhibitor of polyamine biosynthesis, an inhibitor of a different protein kinase, especially protein kinase C, or of a tyrosine protein kinase, such as epidermal growth factor receptor protein tyrosine kinase, an inhibitor of a growth factor, such as vascular endothelial growth factor, a cytokine, a negative growth regulator, such as TGF-β or IFN-β, an aromatase inhibitor, hormones or hormone analogues, and a conventional cytostatic agent.

The disclosed compounds are intended not only for the (prophylactic and, preferably, therapeutic) treatment of human beings, but also for the treatment of other warm-blooded animals, e.g., of commercially useful animals, e.g., rodents, such as mice, rabbits or rats or guinea pigs.

In the groups of preferred compounds of formula (I) mentioned below, definitions of substituents from the above-mentioned general definitions may expediently be used, e.g., in order to replace more general definitions by definitions that are more specific or, especially, by definitions that are indicated as being preferred; preference is in each case given to the definitions indicated above as being preferred or mentioned by way of example.

Special preference is given to such compounds, wherein Y is phenyl that is substituted in the 4position by t-butyl or trifluoromethyl.

Particularly important compounds of the formula (I) include:

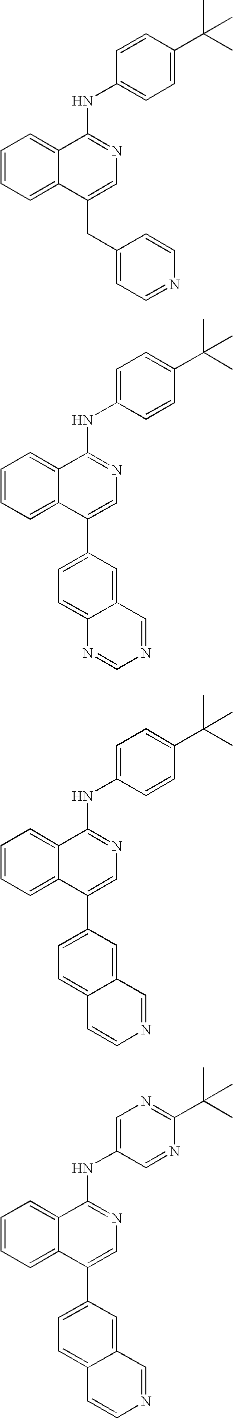

The compounds according to the invention can be prepared by processes known in the art perse for other compounds, especially as described in U.S. Published Application Ser. No. 2002-0010191.

In another aspect, a method is provided for monitoring the efficacy of treatment of a disease characterized by an aberrant MAP kinase signaling pathway in a patient by administration of a Raf kinase inhibitor. The method comprises:
a) measuring the level of phosphoiylation of at least one phosphoprotein identified in Table 1 or 2 in a biological sample obtained from the patient prior to treatment;
b) measuring the level of phosphorylation of the nhosphoprotein +one or more post-treatment biological samples obtained from the patient; and
c) comparing the level of phosphorylation of the phosphoprotein in the sample obtained prior to treatment with the sample(s) obtained post-treatment, wherein a decrease in the level of phosphorylation of the phosphoprotein in the biological sample obtained post-treatment relative to the level of phosphorylation of the phosphoprotein in the biological sample obtained prior to treatment is indicative of the efficacy of the treatment.

The level of phosphorylation of the pre-and post-treatment samples can be measured simultaneously or at different times depending on the method utilized to detect the level of phosphorylation of the phosphoprotein.

In one aspect measurement of the level of phosphorylation may be achieved utilizing antibodies specific to a protein selected from Table 1 or 2.

In another aspect, the invention also includes methods for detecting the presence of a polypeptide or nucleic acid in a sample selected from Table 1 or 2 from a mammal, e.g., a human, by contacting a sample from the mammal with an antibody which selectively binds to one of the herein described polypeptides, and detecting the formation of reaction complexes including the antibody and the polypeptide in the sample. Detecting the formation of complexes in the sample indicates the presence or amount of the polypeptide in the sample.

Antibodies which bind to a protein selected from Table 1 or 2 can be made by standard techniques for monoclonal and polyclonal antibody preparation. For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed proteins selected from Table 1 or 2 or a chemically synthesized polypeptide or Table 1 or 2. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against proteins from Table 1 or 2 can be isolated from the mammal (e.g., from the blood) and further purified by well known techniques, such as protein A chromatography to obtain the IgG fraction.

Antibodies can also be made using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired ligand. Clones expressing Fv fragments capable of binding to the desired ligand are adsorbed to the ligand and thus separated from the non-binding clones in the library. The binding clones are then eluted from the ligand, and can be further enriched by additional cycles of ligand adsorption/elution. Any of the antibodies of the invention can be obtained by designing a suitable ligand screening procedure to select for the phage clone of interest followed by construction of a full length antibody clone using the Fv sequences from the phage clone of interest and suitable constant region (Fc) sequences described in Kabat et al., Sequences of Proteins of Immunological Interest, Fifth Edition, NIH Publication 91-3242, Bethesda MD (1991), vols. 1-3.

In carrying out the method for monitoring the efficacy of treatment of patients with a Raf kinase inhibitor in patients an increased level of administration of the Raf kinase inhibitor may be desirable to decrease the level of phosphorylation of the phosphoprotein detected in a post-treatment sample of the patient to lower levels than detected, i.e., to increase the effectiveness of the Raf kinase inhibitor. Alternatively; decreased administration of the Raf kinase inhibitor may be desirable to increase phosphorylation of the protein to higher levels than detected, i.e., to decrease the effectiveness of the Raf kinase inhibitor.

The method for monitoring the efficacy of treatment of a patient having a disease characterized by an aberrant MAP kinase signaling pathway by administration with a Raf kinase inhibitor can further comprise administering the Raf kinase inhibitor with one or more antiproliferative, cytostatic or cytotoxic compounds as described above.

The efficacy of different Raf kinase inhibitors can be screened and compared using in vitro cellular systems. In one embodiment, a method for monitoring the efficacy of treatment with a Raf kinase inhitor in vitro is provided which comprises:
a) exposing a sample of cells comprising at least one of the phosphoproteins identified in Table 1 or 2 to the Raf kinase inhibitor; and
b) comparing the level of phosphorylation of the at least one phosphoprotein in the sample of cells with the level of phosphorylation of the phosphoprotein in a control sample of cells (without treatment), wherein a decreased level of phosphorylation in the treated sample of cells as compared to the level of phosphorylation in the normal sample is indicative of the efficacy of the Raf kinase inhibitor.

Thus, in one aspect the invention pertains to cells or host cells into which a recombinant expression vector expressing a protein from Table 1 or 2. Suitable host cells for use in this vitro screen include e.g., cancer cell lines or isolated cancer cells such as hepatoma cells, Saos-2 (a human sarcoma cell line), Jurkat (keukemic T-cell line) and line), HeLa (a human cervical cancer cell line), TIL lines (obtained from melanoma patients) and MDA 231 (breast cancer cell line).

In one aspect, a nucleic acid encoding the proteins selected from Table 1 or 2 of the invention is expressed in mammalian cells using a mammalian expression vector. A recombinant mammalian expression vector is capable of directing expression of the nucleic acid preferentially in a particular cell type (e.g., tissue-specified regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Baneaji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (lund et al. (1985) Science 230:912-916), and mammary gland-specific promoters (e.g., milk whey promoter, U.S. Pat. No. 4,873;316 and European Application Publication No. 264,166). Developmentally-regulated promoters are also encompassed, e.g., the murine hox promoters (Kessel and Gruss (1990) Science 249:374-379) and the .alpha-fetoprotein promoter (Campes and Tilghman (1989) Genes Dev 3:537-546).

Vector DNA can be introduced into prokaryotic or eukaryotic host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, can be used to produce (i.e., express) proteins selected from Table 1 or 2. Accordingly, the invention further provides methods for producing proteins selected from Table 1 or 2 using the host cells of the invention. In one aspect, the method comprises culturing the host cell of invention (into which a recombinant expression vector encoding a protein from Table 1 or 2 has been introduced) in a suitable medium such that the protein is produced. In another embodiment, the method further comprises isolating the protein from the medium or the host cell.

In another aspect, a method is provided for diagnosing a disease characterized by an aberrant MAP kinase signaling pathway in a patient. The method comprises:
a) detecting a level of phosphorylation of at least one phosphoprotein identified in Table 1 or 2 in a test biological sample obtained from the patient; and
b) comparing the level of phosphorylation of the at least one phosphoprotein in the test biological sample with the level of phosphorylation of the phosphoprotein in a normal sample obtained from the patient or from another source (e.g., a sample obtained from a disease-free subject or a pre-established control for which the level of phosphorylation of the protein was determined at an earlier time), wherein a higher level of phosphorylation in the test biological sample as compared to the level of phosphorylation in the normal sample is indicative of the presence of the disease characterized by an aberrant MAP kinase signaling pathway in the patient.

Examples of test biological samples and normal (control) samples for use in the diagnostic method are as described above. In a particularly useful embodiment, the level of phosphorylation of the serine 25 residue or serine 38 residue of Op18, and preferably the serine 25 residue of Op18, is detected by methods well known in the art.

In yet another aspect, a method is provided for monitoring the progression of a disease characterized by an aberrant MAP kinase signaling pathway in a patient. The method comprises measuring a level of phosphorylation of at least one phosphoprotein identified in Table 1 or 2 over time in a biological sample obtained from the patient, wherein an increase in the level of phosphorylation of the phosphoprotein over time is indicative of the progression of the disease in the patient.

The following examples serve to illustrate the invention but do not limit the scope thereof in any way.

### EXAMPLE 1

### Inverse ²H-Labeling Utilizing HCT116 Cell Lysate

A. Cell culture and lysate preparation

Human colorectal cell line HCT116 cells are grown in 6-well plates. Prior to harvesting, the cells are treated with 20 μM of the Raf inhibitor BPMI and DMSO control for 1.5 hours, respectively. Cells are then rinsed with PBS, and lysed for 5 minutes at 4° C. in Doriano lysis buffer with 100 μg/mL Perfabloc/2 μg/mL aprotinin/2 μg/mL leupeptin/1 mM NaVO₄/10 mM NaF. The supernatant of the lysates are collected after centrifugation at 3,000 rpm for 5 minutes. The protein concentration is determined using Bio-Rad reagent, and the lysates are frozen at −80° C. prior to fkrther processing and analysis.

One μL of RNase A (20 mg/mL, Sigma, St Louis, Mo.) and 1 μL of RNase T1 (10 units/mL, Invitrogen, Carlsbad, Calif.) are added to each 1 mL of lysates (total 3 mg of HCT116-DMSO control and 3 mg of HCT116-BPMI, respectively), and incubated at 37° C. for half an hour to degrade RNAs. Proteins are denatured using 6 M guanidine HCl, followed by reduction with 20 mM 1,4-dithio-DL-threitol (DTT) at 58° C. for 40 minutes and alkylation with 40 mM iodoacetamide at room temperature for 30 minutes in the dark. Each protein solution is transferred to a Slide-A-Lyzer (10,000 MW cutoff, Pierce, Rockford, IL) dialysis cassette and dialyzed against 2 to 0 M urea/50 mM ammonium bicarbonate to remove small molecule impurities and buffer exchange to 50 mM ammonium bicarbonate. Proteolysis is carried out using modified, sequencing grade trypsin (Promega, Madison, Wiss.) at a 1:200 trypsin-to-protein ratio (wt:wt) in 50 mM ammonium bicarbonate at 37° C. overnight.

The peptide digests are filtered through Centricon Filters (10,000 MW cutoff, Millipore, Bedford, Mass.) to remove large molecule impurities including detergents. Flow-through (peptides) is collected. Solvent and ammonium bicarbonate are subsequently removed by SpeedVac drying.

B. Methyl Esterification and Inverse Labeling

d0- or d3-methanolic HCl (2M) (methyl esterification reagent) is prepared by adding 160 μL of acetyl chloride to 1 niL of anhydrous d0-methyl alcohol or d3-methyl d-alcohol drop wise while stirring. After 10 minutes, 1 mL of the methyl esterification reagent is added to 1.5 mg of lyophilized peptide mixture. The reaction is performed in parallel to two identical aliquots for every sample, one using d0-reagent and one using d3-reagent, respectively. The reaction is allowed to proceed at room temperature for 30 minutes. The excess reagents are removed by SpeedVac drying. Subsequently the peptide mixtures are reconstituted with water. The inverse labeling is achieved by mixing d0-control with d3-treated (BPMI) and d3-control with d0-treated.

C. IMAC

Enrichment of phosphopeptides is performed on a 2.1×30 mm IMAC column (POROS 20 MC, Applied Biosystems, Foster City, Calif.). Briefly, the column is washed with water, 100 mM EDTA in 1 M NaCl, followed by water and 1% acetic acid. The column is then activated with 100 mM FeCl₃. The SneedVac drive, 1 ,g of the inversely labeled methyl esterified peptide mixture (500 μg each form of d0 and d3) is dissolved in 1% acetic acid in 50% acetonitrile/water, and loaded onto iron-activated IMAC column. The unbound peptides are removed by washing with 1% acetic acid in 50% acetonitrile/water. The bound phosphopeptides are eluted with 2% ammonium hydroxide in 50% acetonitrile/water (pH approximately 9 to 10). Acetic acid is added to neutralize the eluent prior to SpeedVac drying. The phosphopeptide mixture is reconstituted with 0.1% formic acid and analyzed using capillary LC/MS, as described below.

D. Capillary HPLC

An Ultimate capillary/nano HPLC system (LC Packings, San Francisco, Calif.) with a Swichos micro column-switching module (LC Packings, San Francisco, Calif.) is used for analysis. Separation is carried out on a 0.18×150 mm capillary column, packed with 3 μm C18 stationary phase of 300-Å pore size (PepMap, LC Packings, San Francisco, Calif.), operating at a flow rate of 2 μL/min. Mobile phase A consists of 0.1% (v/v) formic acid in water and mobile phase B of 0.1% (v/v) formic acid in acetonitrile. Prior to use, the mobile phase is filtered through a 0.22 μm membrane filter (Millipore, Bedford, Mass.) and continuously purged with helium during operation. A FAMOS micro autosampler with a 20 μL sample loop (LC Packings, San Francisco, Calif.) is used for sample injection.

Ten μL of each sample, containing peptides from 100 μg or 150 μg of starting material, is loaded onto a C18 trap column (0.3×5 mm, LC Packings, San Francisco, Calif.). The peptides are first washed with 0.1% formic acid at 20 μL/min. for 3 minutes, then eluted onto the capillary LC column using 5% acetonitrile at 2 μL/min., followed by a gradient from 5-40% B in 60 minutes to elute peptides from the LC column into the Qtof MS for detection.

E. Mass spectrometry—Qtof MS/MS

MS analysis is performed on a Qtof Ultima Global quadruple-time-of-flight mass spectrometer (Micromass, UK) equipped with a Z spray inlet. On-line coupling of capillary LC to Qtof was through a nanospray interface (Micromass, UK) using a 20 μm i.d. fused silica capillary as electrospray emitter. For MS/MS analysis, the data-dependent acquisition mode (automatic switching from MS mode to MS/MS mode based on precursor ion's intensity and charge state) is used. It involves one positive mode MS survey scan followed by MS/MS on the five most abundant multiply-charged ions.

Database Searching

The resulting MS/MS spectra are used to search NCBInr protein database using MASCOT program (Matrix Science, UK). In these searches, static modification of 14 Da to Glu, Asp and C-terminus is selected. Phosphorylation on Ser, Thr and Tyr is considered variable modifications. By comparing the experimental MS/MS spectra with a database of theoretical peptide fragments and by utilizing an appropriate scoring algorithm, the closest match, containing information to assign not only the sequence, but also the site of phosphorylation and the identity of phosphoprotein, is expected to be identified from the database search. For all sequence reported, spectra are verified manually.

Stable isotope labeling is achieved at the time of methyl esterification. The differential labeling with one sample reacted with methanol and the other with d3-methanol allows for the quantitative comparison of two phospho-profiles for information of phosphorylation changes.

### EXAMPLE 2

Application of the Inverse Labeling Method to Cellular Studies Utilizing the Raf-Inhibitor BPMI

Cell lysates are treated without and with the Raf inhibitor, BPMI. The DMSO control and Raf inhibitor-treated HCTI 16 cell lysates are processed, digested and methyl esterified in the inverse labeling fashion. One mg each of the two inversely-labeled peptide mixtures (decontrol mixed with d3-treated, and d3-control mixed with d0-treated, 500 μg each form) is purified by IMAC. Approximately 30% of each IMAC enriched phosphopeptide mixture is then analyzed using capillary LC/MS. For quality control purposes, a 0.5% β-casein phosphoprotein is added to each sample prior to sample preparation and serves as an internal standard to QC the entire process of lysate preparation, methyl esterification, IMAC purification and LC/MS analysis.

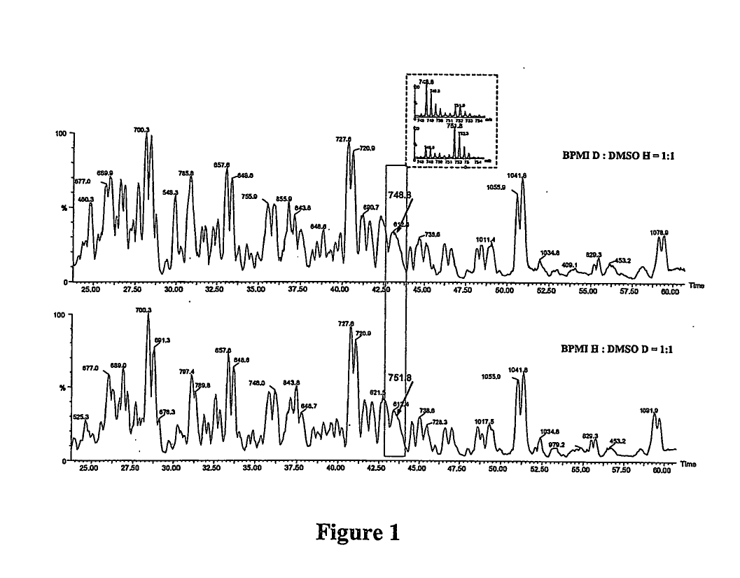

FIG. 1 illustrates the LC/MS chromatograms obtained from the inverse labeling-MS analysis of IMAC enriched phosphopeptides from the study. As expected, doubly- and triply-charged peptide ions at m/z 1080.5/1091.0 and 720.6/727.6, corresponding to the methyl ester of β-casein phosphopeptide FQpSEEQQQTEDELQDK (SEQ ID NO:_) and its isotopic analogue, are detected in every sample with chromatographic peak heights between the light and heavy isotopic pairs all within 10% variation, suggesting consistent recovery of phosphopeptides from each lysate samples. Initial data analysis reveal more than 500 isotopic pairs of phosphopeptides. Although most of them are found to be doublets of approximately the same intensity, indicating similar levels of phosphorylation between the treated and the control cell lysates, 11 phosphopeptides are found to show an inverse labeling pattern characteristic for down-regulation upon the BPMI treatment (see Table 1). The sequence and the site of phosphorylation is defined for five of them.

TABLE 1

Phosphorylation Changes in HCT116 Cells
Upon BPMI Treatment

Change

| | |
|---|---|
| Sequence | (treated/ |

| | | | |
|---|---|---|---|
| ID NO: | Phosphopeptide | Phosphoprotein | control) |

| | | |
|---|---|---|
| ASGQAFELILpSPR | Oncoprotein 18 | 0.4 |
| | | (60% down) |
| SKESVPEFPLpSPPK | Oncoprotein 18 | 0.6 |
| | | (40% down) |
| LPS*pSPVYEDAASFK | Oncogene EMS1 | 0.8 |
| | | (20% down) |
| T*QpTPPVpSPAPQPT | Oncogene EMS1 | 0.7 |
| EER | | (30% down) |
| IEpSPKLER | Heat-shock 110 | 0.7 |
| | kD protein | (30% down) |

S*, T* = possible phosphorylation sites

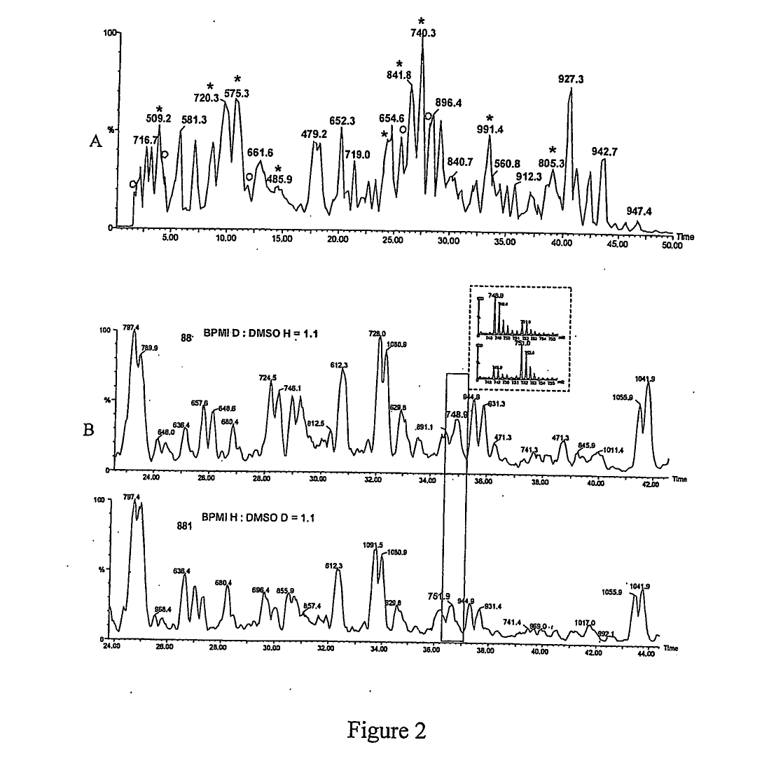

Among the peptides identified of down-regulation in phosphorylation upon BPMI treatment, a consensus sequence is evident around the phosphorylation sites (pSer-Pro), which strongly suggested that they are mechanism-based changes and implicate the significance of the results. One phosphopeptide which is detected to be significantly down-regulated in the drug-treated cells, as shown in the inset of FIG. 2, is identified as from oncoprotein 18 (Sathmin) of serine²⁵ phosphorylation. Previous studies show that Ser²⁵ of Op18 is a major substrate for the mitogen-activated protein (MAP) kinase, a down-stream kinase in the Raf pathway (see Marklund et al., J BioL Chem., Vol. 268, pp. 15039-15047 (1993)). More importantly, good quantitative correlation is observed between Ser²⁵ phosphorylation of Op18 (measured by MS) and MEK kinase phosphorylation (measured by anti-phosphoMEK antibody and Western Blot) in several experiments (data not shown). MEK is the down-stream kinase of Raf in the Rafpathway.

The number of carboxyl groups of a phosphopeptide can be readily calculated according to the mass difference of an isotopic pair. This information of the number of acidic residues in a sequence can be used to further verify the phosphopeptide sequence assignment from the database search (using MS/MS).

### EXAMPLE 3

Application of Inverse Labeling Method to an In Vivo Study of the Raf Inhibitor BPMI: Tumor Tissue DU145 Analysis

The phosphoproteome mapping method is further tested/applied to an in vivo study of the Raf inhibitor, BPMI (one-hour treatment, 200 mg/kg p.o.), in the analysis of tissue lysates of mouse tumor xenograft. Direct analysis of the tryptic digest of the lysates reveals dominant signals from mouse serum albumin and hemoglobin (see FIG. 2A), likely from the blood in the tissue.

The methyl esterification/IMAC procedure is successful in removing the blood contamination from the tissue samples. As shown in FIG. 2B, inverse labeling-MS analysis after IMAC enrichment clearly detects the down-regulation of Ser²⁵ phosphorylation of oncoprotein 18, confirming the finding of the cellular studies. Additional changes in phosphorylation are also detected which includes oncogene EMS1, mouse fetuin and Epithelial-cadherin (see Table 2).

TABLE 2

Phosphorylation Changes in Tumor Tissues
Upon 1 Hour BPMI Treatment

Change
(treated/

| | | |
|---|---|---|
| Phosphopeptide | Phosphoprotein | control) |

| | | |
|---|---|---|
| ASGQAFELILpSPR | Oncoprotein 18 | 0.4 |
| | | (60% down) |
| SKESVPEFPLpSPPK | Oncoprotein 18 | 0.8 |
| | | (20% down) |
| LPS*pSPVYEDAASFK | Oncogene EMS1 | 0.7 |
| | | (30% down) |
| T*QpTPPVpSPAPQPTEER | Oncogene EMS1 | 0.6 |
| | | (40% down) |
| VoxMHTQCHSTPDpSAEDV | Mouse fetuin | 0.6 |
| R (SEQ ID NO _) | | (40% down) |
| MRDWVIPPIpSCPENEK | Epithelial-cadherin | 0.5 |
| (SEQ ID NO_) | (mouse/human) | (50% down) |

S*, T* = possible phosphorylation sites

It will be understood that various modifications may be made to the embodiments and/or examples disclosed herein. Thus, the above description should not be construed as limiting, but merely as exemplifications of preferred embodiments. Those skilled in the art will envision other modifications within the scope and spirit of the claims appended hereto.

### EXAMPLE 4

Western Blot Analysis of Raf-inhibitor-treated HCT116 Cell Lysates

HCT1 16 cells are prepared. as in Example 1 above and treated with a Raf inhibitor (BPMI) for 1.5 hr. Cells are lysed in Incomplete Laemmli buffer (0.1 IM Tris.Cl pH 7.0, 4% SDS, 10% glycerol), and passed through a syringe fitted with 25 gauge needle to break apart genomic DNA. The lysate is boiled for 5 minutes, and loaded onto 10% SDS-PAGE gel. Proteins in the SDS-PAGE gel are then transferred to nitrocellulose membrane. The membrane is blocked with 5% milk for 1 hr, and incubated with the an antibody specific for Ser²⁵ phosphorylated OP18 for an additional hour. The phosphor-Ser²⁵ specific OP18 antibody is raised in rabbit using standard techniques and purification known in the art. The amino acid sequence used to generate phosphor-Ser²⁵ specific OP18 antibody is ELILpSPRSKESVPEFP (SEQ ID NO:_). The blot is washed three times with TBST 15 minutes each, and then incubated with anti-rabbit HRP-conjugated antibody for 1 hr. The blot is washed three times with TBST 15 minutes each, and the antibody bound proteins are labeled using SuperSignal West Dura Extended Duration substrate (Piece Biotechnologies, Rockford, Ill.). The phosphorylated OP18 proteins are detected via exposing the blot through a film (Eastman Kodak, Rochester, N.Y.).

The treatment of HCT116 cells with BPMI leads to the inhibition of OP18 phosphorylation at Ser²⁵ residue (data not shown). At 10 μM, almost no phosphorylation is seen at Ser²⁵ residue of the OP18 protein. The IC50 value of BPMI for inhibition of MAPK pathway in HCT116 cells is 5.3 μM. Additional data also provides that BPMI inhibits OP18 Ser²⁵ phosphorylation with an IC50 of approximately 5 μM. Thus, it appears that both MAPK and OP18 Ser²⁵ phosphorylation are inhibited at a similar level for a given dose of a raf inhibitor.

1. A method for treating a patient having a disease characterized by an aberrant MAP kinase signaling pathway, the method comprising identifying a patient having the disease by detecting an increased level of phosphorylation of at least one phosphoprotein identified in Table 1 in a biological sample obtained from the patient, and administering to the patient an effective amount of a Raf kinase inhibitor.

2. The method of claim 1, wherein the disease characterized by an aberrant MAP kinase signaling pathway is a cancer.

3. The method of claim 2, wherein the cancer is selected from the group consisting of cancer cell is selected from the group consisting of a melanoma, a colorectal cancer, an ovarian cancer, a glioma, an adenocarcinoma, a sarcoma, a breast cancer, a lung cancer and a liver cancer.

4. The method of claim 3, wherein the cancer is a melanoma.

5. The method of claim 1, wherein the level of phosphorylation of the serine 25 residue or serine 38 residue of Op18 is detected.

6. The method of claim 1, wherein the level of phosphorylation of the serine 25 residue of Op18 is detected.

7. The method of claim 1, wherein the biological sample is obtained from a cell or cells, a tissue or tissues, blood, serum, stool, urine, sputum, amniotic fluid or a bone tissue biopsy.

8. The method of claim 7, wherein the biological sample is skin cells.

9. A method for monitoring the efficacy of treatment of a disease characterized by an aberrant MAP kinase signaling pathway in a patient by administration of a Raf kinase inhibitor, the method comprising:
a) measuring the level of phosphorylation of at least one phosphoprotein identified in Table 1 in a biological sample obtained from the patient prior to treatment;
b) measuring the level of phosphorylation of the phosphoprotein in another biological sample obtained from the patient post-treatment; and
c) comparing the level of phosphorylation of the phosphoprotein in both samples, wherein a decrease in the level of phosphorylation of the phosphoprotein in the biological sample obtained post-treatment relative to the level of phosphorylation of the phosphoprotein in the biological sample obtained prior to treatment is indicative of the efficacy of the treatment.

10. The method of claim 9, wherein the disease characterized by an aberrant MAP kinase signaling pathway is cancer.

11. The method of claim 9, wherein the cancer is selected from the group consisting of a melanoma, a colorectal cancer, an ovarian cancer, a glioma, an adenocarcinoma, a sarcoma, a breast cancer, a lung cancer and a liver cancer.

12. The method of claim 9, wherein said measuring step utilizes an antibody.

13. The method of claim 12 wherein said antibody binds to phosphorylated Oncoprotein 18.

14. A method for diagnosing a disease characterized by an aberrant MAP kinase signaling pathway in a patient, the method comprising:
a) detecting a level of phosphorylation of at least one phosphoprotein identified in Table 1 in a test biological sample comprising the at least one protein obtained from the patient; and
b) comparing the level of phosphorylation of the at least one phosphoprotein in the test biological sample with the level of phosphorylation of the phosphoprotein in a normal sample obtained from the patient or from another source, wherein a higher level of phosphorylation in the test biological sample as compared to the level of phosphorylation in the normal sample is indicative of the presence of the disease characterized by an aberrant MAP kinase signaling pathway in the patient.

15. The method of claim 14, wherein the disease characterized by an aberrant MAP kinase signaling pathway is a cancer.

16. The method of claim 15 wherein the cancer is selected from the group consisting of cancer cell is selected from the group consisting of a melanoma, a colorectal cancer, an ovarian cancer, a glioma, an adenocarcinoma, a sarcoma, a breast cancer, a lung cancer and a liver cancer.

17. The method of claim 15, wherein the cancer is a melanoma.

18. The method of claim 14,wherein the level of phosphorylation of the serine 25 residue or serine 38 residue of Op18 is detected.

19. The method of claim 14, wherein the level of phosphorylation of the serine 25 residue of Op18 is detected.

20. The method of claim 14, wherein the biological sample is obtained from a cell or cells, a tissue or tissues, blood, serum, stool, urine, sputum, amniotic fluid or a bone tissue biopsy.

21. The method of claim 20, wherein the biological sample is skin cells.

22. A method for monitoring the progression of a disease characterized by an aberrant MAP kinase signaling pathway in a patient, the method comprising measuring a level of phosphorylation of at least one phosphoprotein identified in Table 1 over time in a biological sample obtained from the patient, wherein an increase in the level of phosphorylation of the phosphoprotein over time is indicative of the progression of the disorder in the patient.

23. The method of claim 22, wherein the disease characterized by an aberrant MAP kinase signaling pathway is a cancer.

24. The method of claim 23, wherein the cancer is selected from the group consisting of cancer cell is selected from the group consisting of a melanoma, a colorectal cancer, an ovarian cancer, a glioma, an adenocarcinoma, a sarcoma, a breast cancer, a lung cancer and a liver cancer.

25. The method of claim 22, wherein the level of phosphorylation of the serine 25 residue or serine 38 residue of Op18 is detected.

26. The method of claim 22, wherein the level of phosphorylation of the serine 25 residue of Op18 is detected.
